(19) United States
(12) Patent Application Publication
(10) Pub. No.: US 2013/0040902 A1
(43) Pub. Date: Feb. 14, 2013

(54) METHOD FOR ENHANCING MILK QUANTITY AND QUALITY

(71) Applicants: Joshua Miron, Bet-Dagan (IL); Ariel Shabtay, Ramat Yishay (IL); Eli Budman, Moshav Bat Shlomo (IL); Gilad Agmon, Ramat Hasharon (IL)

(72) Inventors: Joshua Miron, Bet-Dagan (IL); Ariel Shabtay, Ramat Yishay (IL); Eli Budman, Moshav Bat Shlomo (IL); Gilad Agmon, Ramat Hasharon (IL)

(73) Assignee: GANIR LTD., M.P. Sde Gat (IL)

(21) Appl. No.: 13/642,385

(22) PCT Filed: Apr. 17, 2011
(86) PCT No.: PCT/IL2011/000327

§ 371 Date: Oct. 19, 2012

Related U.S. Application Data
(60) Provisional application No. 61/326,271, filed on Apr. 21, 2010.

(51) Int. Cl. A61K 31/7048; A61P 39/06; A61P 29/00; A61P 15/00; A61P 3/00; A61P 31/04

(52) U.S. Cl. 514/23

(57) ABSTRACT

This invention provides a method for enhancing quantity and quality of milk in mammals, while improving their health. The method particularly enables to substantially increase global milk production and improve economy of the dairy industry, without negatively affecting the wellbeing of dairy cattle.

### FIELD OF THE INVENTION

The present invention relates to a food additive comprising pomegranate, and to a method for enhancing the production of milk in dairy industry, while increasing the milk quality and well being of the animals.

### BACKGROUND OF THE INVENTION

Milk of the ruminants has had an important role in human diet, throughout history, as a source of nearly all essential nutrition components. The world production of bovine milk is more than 500 billion liters per year, and even a small increase in the milk yield contributes immensely to the feeding of humankind, particularly in developing countries. However, the increase should not be at the expense of either milk quality or animals' well being, as it often happened when introducing new techniques in the dairy industry. The modern society is increasingly sensitive to suffering of livestock, and animal's well being must not be damaged by new manufacturing procedures. Moreover, the decrease of useful components or the appearance of undesired materials is followed up still more strictly in modern quality control, the undesired materials including antibiotics, somatic cells, etc. It is therefore an object of this invention to provide a method for increasing the production of milk and improving its quality without causing undesired effects on the livestock.

It is also an object of this invention to provide a method for increasing the production of milk without negatively affecting the animals well being.

It is further an object of the invention to provide a food additive for enhancing the milk quantity and quality in mammals.

Other objects and advantages of present invention will appear as description proceeds.

### SUMMARY OF THE INVENTION

This invention provides a method for enhancing the quantity and quality of milk in a mammal, while maintaining or enhancing its health, comprising feeding said mammal with a ration comprising an additive derived from pomegranate plant. In a preferred embodiment of the invention, said mammals are ruminants. Said additive is derived from a pomegranate part selected from the group consisting of leaves, flowers, fruits, fruit cores, fruit peels, fruit pulp membranes, and fruit arils, or mixtures thereof. In one aspect of the invention, said additive comprises an extract of said pomegranate parts, or a powder based on said pomegranate parts. In an important embodiment of the invention, said additive comprises an active compound, for example antioxidant, in a concentration higher than originally present in said pomegranate part before processing the part into the desired additive. The pomegranate active components, present in the pomegranate parts before their processing to said additive, are preferably preserved and even concentrated in said additive relatively to said parts. In another aspect of the invention, said additive is a silage comprising said pomegranate parts. In a preferred embodiment of the invention, provided is a method for enhancing the yield of milk obtained from ruminants, comprising adding to the ration for said ruminants an additive comprising dry peels or an extract of pomegranate peels or an ensilage of said peels. Said ruminants may include sheep, goat, and other animals. In an important aspect of the invention, the ruminants are dairy cattle. Said extract comprises from 1 to 100 wt % of dry mass. In a preferred embodiment of the invention, the content of antioxidants in the obtained milk is enhanced by adding said part of the pomegranate plant, or its extract or a derivative, or a silage comprising said part, to the ration of the animal. The milk production in said mammal, particularly ruminant, is increased by 1.5% or more after including said additive in said ration. More preferably, said milk yield is increased by 3.0% or more. Still more preferably, said milk yield is increased by 6.0% or more. Said adding the extract to ration preferably results in lowering the number of somatic cells in the obtained milk. The daily milk production in a mammal may increase by 11% or more, for example by 15%. The milk production may typically increase, according to various conditions and state and health of the animals, between 2 and 11%. The effect and efficacy of the feed additive may vary according to the health conditions of the animals, and according to the specific time of administration: before or after calving, during lactation o period, before, during or after different types of stress (environmental, physiological or oxidative stress, for example). The effect and efficacy of the feed additive may vary according to the mode of administration: continuously, intermittently, different doses according to the different physiological states or health of the ruminant. The method of the invention enhances resistance of the mammal toward stress and infection. For example, mammal's resistance and health are enhanced in critical periods of pregnancy, delivery, and lactation, affecting positively both the mother and the young ones. Feeding the animal, in accordance with the method of the invention, with ration comprising a pomegranate part, or extract or silage comprising pomegranate part, results in improving animals' well being and health, and lowers morbidity, for example morbidity associated with bacterial infections, mastitis, uterus inflammation, ketosis, etc. The method of the invention, directed to enhancing the quantity and quality of milk in a mammal and comprising feeding said mammal with ration containing an additive derived from pomegranate plant, results in improving at least one of the parameters selected from the number of somatic cells in milk, antioxidative activity of milk, milk composition, voluntary dry matter intake, dry matter and NDF digestibility of said ration, body mass changes, udder health, general health of the animal, the rumination time, and the recumbence time.

The invention provides a method for improving efficiency of the milk production in dairy industry, comprising feeding milking ruminants with a fodder consisting of ration, and a pomegranate-derived additive, such as peel extract or a material derived from pomegranate peels, increasing the daily milk yield of said ruminants, improving the milk quality, and enhancing health and well being of said ruminants.

In an important aspect, the methods of the invention enhance health and well being of said ruminants under stress conditions selected from ambient heat load, beginning of lactation, bacterial infection, inflammation of uterus, and inflammation of udder.

The invention relates to a food additive for increasing the milk yield in ruminants, comprising a material derived from pomegranate parts selected from leaves, flowers, fruits, fruit cores, fruit peels, fruit pulp membranes, fruit arils, and mixtures thereof. Said material is preferably derived from pomegranate parts by an extraction procedure, or by drying, or by ensiling. Said material may be derived from said pomegranate parts by a chemical or biological process, possibly comprising separation techniques, fermentation techniques, silaging, application of enzymes, etc. Said additive preferably comprises an aqueous or alcoholic extract of various parts of pomegranate fruit, such as leaves and/or flowers, mainly fruit peels or parts comprising the peels, or other plant parts, or it comprises a powder based on pomegranate parts or said extracts. Preferably, said additive comprises a concentrated aqueous extract from pomegranate peels. Said pomegranate parts may be processed to obtain said additive by including, in various order, the steps of drying, extracting, treating with enzymes, fermenting, and ensiling.

In one embodiment, the invention provides an enriched fodder for a mammal, particularly ruminant, comprising a suitable ration and an additive comprising a concentrated aqueous extract from pomegranate peels, the additive being in an amount of from 0.5 to 5 weight parts of said concentrated extract per 100 dry weight parts of said ration. In other preferred embodiment, the invention provides an enriched fodder for a mammal, particularly ruminant, comprising a suitable ration and an additive comprising ensiled pomegranate peels, the additive being in an amount up to 20 weight parts of ensilage per 100 dry weight parts of said ration. In other embodiment, dry peels or other derivatives of pomegranate parts may be employed, and the additive may constitute from 0.5 to 10 weight parts or more per 100 dry weight parts of said ration. When a material comprising pomegranate peels is related to, either separated peels are intended or pomegranate fruit parts comprising said peels, such as squeezed fruits.

In one embodiment of the invention, the additive comprises an aqueous extract of 5 Brix containing punicalagins A+B in an amount of more than 2,000 mg/Kg, total phenolics determined by Folin-Ciocalteu test in an amount of more than 10,000 mg/Kg, and ORAC value of more than 5,000 μm TE/100 g.

In a preferred embodiment, the food additive of the invention comprises at least one characteristic selected from punicalagins A+B in an amount of more than 10,000 mg/Kg, total phenolics determined by Folin-Ciocalteu test in an amount of more than 40,000 mg/Kg, and ORAC value of more than 20,000 TE/100 g.

Also provided is a process for manufacturing said additive, which in one embodiment comprises milling the pomegranate peels, mixing with a solvent acceptable in animal husbandry, thereby obtaining a suspension of insoluble fragments in liquid, separating said suspended fragments from said liquid, and removing at least a part of said solvent from the mixture. Said solvent may comprise water, alcohol, or a mixture thereof, and said separating may comprise sieving, filtering, or centrifuging. Said process typically further comprises determining at least one characteristic selected from the group consisting of punicalagins, total phenolics, ORAC value, and ellagic acid, while employing measuring techniques and units according to the accepted standards. In other embodiments, said process comprises ensiling or fermenting of pomegranate plant parts.

### BRIEF DESCRIPTION OF THE DRAWINGS

The above and other characteristics and advantages of the invention will be more readily apparent through the following examples, and with reference to the appended drawings, wherein:

FIG. 1. Shows the effect of the instant method on the antioxidant activity of milk; four reaction mixtures represent the control glucose oxidase (GO) mixture, standard antioxidant mixture of epicatechin, milk of the control cow group, and milk obtained by the method of the invention;

FIG. 2. is Table 1, showing the effects of adding 4% pomegranate peels water extract to ration on milking performance and health of dairy cows during late pregnancy and 80 days after calving;

FIG. 3. is Table 2, showing the effects of adding 4% pomegranate peels water extract on milking performance and health of dairy cows identified by low somatic cells count (SCC<150,000/ml milk) at the onset of the experiment;

FIG. 4. is Table 3, showing the effects of adding 4% pomegranate peels water extract on milking performance and health of dairy cows identified by high somatic cells count (SCC>150,000/ml milk) at the onset of the experiment; and

FIG. 5. is Table 4. showing the effects of adding various amounts of pomegranate peels water extract to ration on milking performance, antioxidative activity in milk, and health of lactating cows.

### DETAILED DESCRIPTION OF THE INVENTION

It has now been found that the addition of an aqueous extract from pomegranate peels increased the milk yield in dairy cows by more than 7%, while decreasing the amount of somatic cells in the milk. Under various conditions, the daily yield of milk per cow increased by three liters.

It has further turned out that the addition of a concentrated extract from pomegranates (CEP) to cows' fodder, and particularly CEP from peels, increased the antioxidant activity of their milk by more than 20%. Extracts from pomegranate fruit or components thereof, particularly peels, have been found to contain high amounts of active components like polyphenols and bioflavonoids. The alcoholic or water extracts have antimicrobial and antifungal effects. Simultaneously, animals' well being has been improved, as exhibited by lower morbidity, longer periods of rumination, and longer periods of recumbence. The milk yield has substantially increased. Other economic and technical parameters characterizing the milk production have improved or remained unchanged, comprising protein and fat content, body weight change, dry matter and fiber intake and digestibility, and feed utilization efficiency.

The invention, thus, relates to a process for manufacturing milk, particularly ruminants' milk, comprising adding to ruminants' fodder a pomegranate component, said process increasing the milk quantity and quality, while improving animals' health and well being; the increase of milk quality comprises enhancing the antioxidant activity and reducing the number of somatic cells in milk, while the improvement of animals' health comprises lowering the number of acute infections, improving the udder state, and particularly enhancing health of calving cows. Cows before giving birth and during the month after it are especially vulnerable, and prone to infection, and the addition of a pomegranate extract like CEP improves the animals resistance and contributes to their general health, which positively reflects on their subsequent milk production. The inclusion of phenolic materials from pomegranates in ration for lactating cows may reduce methane production in the rumen, further contributing to animals' comfort. Moreover, such a reduction in the methane production would also conform to the increasing concern about huge amounts of methane emitted to the atmosphere by dairy industry which employs more than one billion cattle.

The experiments (see, for example, Tables 1-4) showed that the cows fed with pomegranate extract enjoyed better health than the control group. The increased milk yield is, thus, attained without burdening the animals with new milking or other instruments, or without overloading their metabolism with new chemical agents, such as antibiotics or hormones.

The invention provides a method for enhancing the yield of milk obtained from ruminants, comprising adding to the ration for said ruminants an extract of various parts of pomegranate fruit, leaves and/or flowers, mainly fruit peels or powder based on said pomegranate parts. Such an extract is preferably obtained from pomegranate peels using aqueous-based or alcoholic extraction, followed by concentrating the extract so that the desired dry weight content is attained. In one embodiment of the invention, a method for the preparation of pomegranate extract is provided, which comprises milling the pomegranate peels, mixing with water or alcohol or a mixture thereof, sieving or filtering or centrifuging, and removing a part of the solvent from the mixture by known methods like evaporation. In some embodiments the liquid extract may be subjecting to ultra filtration and/or resin processes. The temperature of the operations may be, for example, between 4° C. and 80° C. In other aspects, a method for preparing a pomegranate peel extract comprises a fermentation step. A person skilled in the art may incorporate known techniques when processing the peels to ensure in the final extract a sufficient concentration of the desired pomegranate components. As the administration of some pomegranate components in excess may adversely affect the well being of the livestock, it is useful to analyze and standardize the feed additive according to the active compounds. Parameters to be possibly checked, without limitation to these, include total polyphenols, condensed and hydrolysable tannins, punicalagin, punicalin, ellagic acid, gallic acid, punicic acid, phytochemicals, ellagitannins, catechins, procyanidins, anthocyanins, anthocyanidins, flavonols, sterols, terpenoids, antioxidant activity.

In one embodiment of the invention, a process for manufacturing a pomegranate component-comprising extract comprises a step of mixing a pomegranate component with a solvent containing alcohol. In a preferred embodiment, ethanol is employed for extracting dry or wet pomegranate fruit parts, particularly peels, for example with 95% ethanol. Other mixtures of ethanol and water may be employed.

In one preferred embodiment of the invention, the residual matter after squeezing juice from healthy pomegranate fruits is mixed with water, for example three weight parts of water per one weight part of the pomegranate matter, and after a soaking period of several hours the water extract is separated from the insoluble suspended fragments by filtration or centrifugation and concentrated in a vacuum evaporator, for example to a final mass of less than the mass of said residual matter. In food industry, there are various parameters used for characterizing antioxidants or polyphenolic materials in food products, and several of them may be chosen for assessing how the desired materials get concentrated in the final concentrated extract relatively to the concentrations in the original fruit or fruit parts. Said parameters may comprise, for example, oxygen radical absorbance capacity (ORAC) expressed as trolox equivalents, the amount of punicalagins, the amount of ellagic acid, total phenolics determined by Folin-Ciocalteu reagent expressed as gallic acid, etc.

An extract prepared in accordance with the invention and to be added to animal's fodder may comprise, for example, punicalagins A+B in an amount of more than 10,000 mg/Kg, and/or total phenolics determined by Folin-Ciocalteu in an amout of more than 40,000 mg/Kg, and/or ORAC value of more than 20,000 μm TE/100 g. In a preferred embodiment of the invention, said extract may comprise punicalagins A+B in an amount of more than 20,000 mg/Kg, and/or total phenolics determined by Folin-Ciocalteu in an amout of more than 80,000 mg/Kg, and/or ORAC value of more than 40,000 TE/100 g.

In a preferred embodiment of the invention, an additive prepared in accordance with the invention and to be added to animal's fodder contains at least one of the following components in the following concentrations: punicalagins A+B in an amount of more than 2,000 mg/Kg, total phenolics determined by Folin-Ciocalteu in an amount of more than 10,000 mg/Kg, ORAC value of more than 5,000 μm TE/100 g, and ellagic acid of more than 200 mg/Kg.

In another preferred embodiment of the invention, an additive prepared in accordance with the invention and to be added to animal's fodder contains all of the following components in the following concentrations: punicalagins A+B in an amount of more than 2,000 mg/Kg, total phenolics determined by Folin-Ciocalteu in an amount of more than 10,000 mg/Kg, ORAC value of more than 5,000 μm TE/100 g, and ellagic acid of more than 200 mg/Kg.

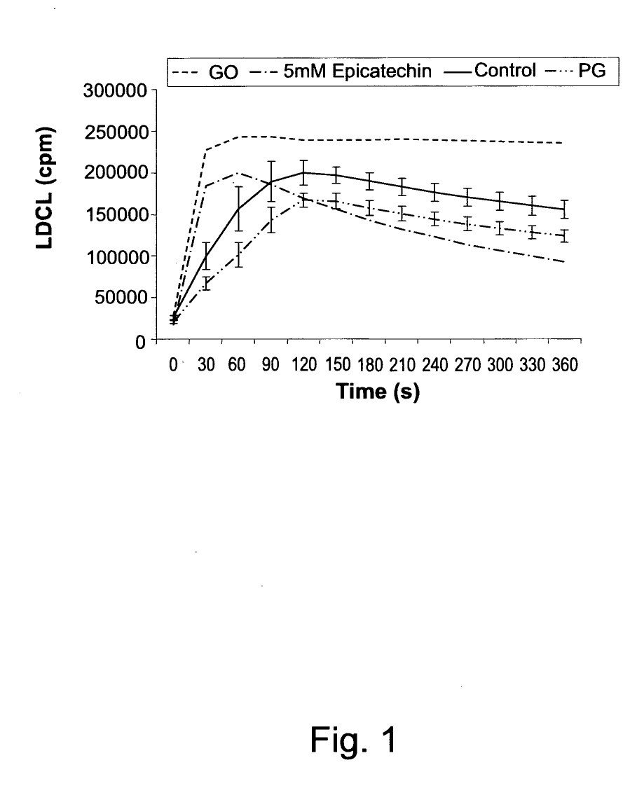

The resulting milk product, obtained after administering CEP to ruminants, has the full quality, and in a preferred embodiment it exhibits increased contents of antioxidants, which are desired components in modern nutrition. The antioxidant activity can be determined, for example, by chemiluminescence methods [see Ginsburg I. et al.: Inflammopharmacology 12 (2004) 305-20]. The cited method showed higher antioxidative activity in milk obtained by the method of the invention (see FIG. 1).

Thus, the invention enables to increase the global milk production, while also improving the milk quality, without compromising animals' well being, and while possibly decreasing the level of undesired emission gases released to the atmosphere.

In one preferred embodiment, the milk production increases by at least 3%, which will bring an immense contribution on the global scale. In other embodiment the average daily milk production increases by 5% or more. In some cows, the average daily milk production increases by up to 10% or more. The invention aims at increasing profitability of the milk production in dairy industry, and at improving the quality of the animals' life. The method of increasing the average daily milk production in cows preferably comprises a step of admixing a pomegranate-comprising component, in a liquid or solid form, to the ration in an amount of between 0.5 and 20 weight parts per 100 weight parts of the ration dry weight. When said pomegranate-comprising component is an extract, it is admixed to the ration in an amount of between 0.5 and 10 weight parts per 100 weight parts of the ration dry weight. In one embodiment, the concentrated peel extract is added in an amount of from 1 to 4 wt % relatively to ration dry weight.

The process for manufacturing a concentrated extract from pomegranate (CEP) component employs mixing an extracting solvent with an extracted component originating from pomegranate, wherein said solvent is preferably selected from alcohols, water, and mixtures thereof, and wherein said extracted component is selected from leaves, flowers, fruits, fruit cores, fruit peels, fruit pulp membranes, and fruit arils (cased seeds). In a preferred embodiment of the extraction process, the extracted component comprises fruit peels, either wet or dried, or fruit parts comprising peels like squeezed fruit. In a preferred embodiment, the extracting solvent is either water or ethanol. A non-limiting example of the process includes the steps of drying the peels, thereby reducing their mass to 15-30%, milling the dried peels, adding ethanol in an amount of 5-10 weight parts per 1 part of the dried peels, for example 95% ethanol, heating the mixture up to a temperature between ambient and 80° C. for a period between 1 and 6 hours, and removing a apart of the alcohol. Another non-limiting example of the process includes the steps of obtaining pomegranate parts comprising peels, such as squeezed fruit after removing juice, adding water in an amount of 2-5 weight parts per 1 part of the pomegranate material, stirring for several hours at a temperature between ambient and 80° C., and removing a greater part of the water by vacuum evaporation. In another embodiment, the process comprises mixing non-dried peels with a solvent. In another embodiment, the process comprises a step of fermentation or ensilage of the pomegranate parts, particularly parts comprising the peels; the product of such fermentation or ensilage may be further processed or directly added to the animal's ration.

The water extract of pomegranate component (CEP), such as peels, added to the fodder contributes to an increase of the milk daily yield from the milking ruminant. Moreover, other parameters characterizing the milk production process and the milk quality are improved, depending on the circumstances, including economically corrected milk yield (ECM, the corrected yield of standard milk with desired content of fat and protein according to milk industry requirements), the number of somatic cells in milk, antioxidative capacity of milk, milk composition, amount of consumed food and corresponding dry matter and fiber digestion, body mass changes, udder health, general health of the animal, the rumination time, and the recumbence time. Said circumstances include the animal breed, breeding conditions, relative time before or after delivery, the presence of infectious factors in the environment, weather, etc. Said milk composition may include the concentrations of protein, fat, lactose, protein/fat ratio, etc. Said fiber digestion may comprise the digestion of dry matter and NDF (Neutral Detergent Fiber). The importance of the fiber content and digestion for the general health of the animals has been long known. The beneficial aspects of feed fiber are due to its effects on chewing, salivation, and rumen function. NDF is a way for describing the fiber, measuring the content of cellulose, lignin and hemicellulose, and it seems to be associated with dry matter intake; dry matter intake is the main factor affecting milk production. Said udder health is usually endangered by mastitis. Said rumination time and recumbence time reflect the animal's comfort and are important for evaluating the well being of the ruminants, particualrly cattle.

The method of the invention for improving the milk production aims at improving animals' health in one aspect, and at increasing the milk quantity and quality in another aspect; however, both aspects are closely interrelated, as a skilled person understands. Under certain conditions, the milk yield is increased relatively more in animals affected by health problems (see, for example, Tables 1 and 3), than in initially healthy animals (Table 2), as the CEP seems to primarily affect the animals' health. Anyway, the method of the invention has a high potential for both increasing the milk production and for improving the animals' well being, and preventing udder infection in healthy cows.

The invention provides fodder for ruminants comprising, in one embodiment, an extract from pomegranates or pomegranate components. In a preferred embodiment of the invention, fodder for milking cows comprises from 0.5 to 5% of a concentrated aqueous pomegranate peel extract, measured as weight of extract concentrate per dry weigh of basic (pomegranate-free) ration. In other embodiments, the fodder for ruminants according to the invention comprises an additive derived from pomegranates or pomegranate components, particularly from 0.5 to 10% of said additive comprising peels, measured as weight of additive per dry weigh of basic (pomegranate-free) ration. In still another embodiment, the invention provides fodder for ruminants comprising an additive derived by ensiling pomegranate components, preferably comprising peels, the fodder comprising from 0.5 to 20% of said additive, measured as weight of additive per dry weigh of basic (pomegranate-free) ration.

In one preferred embodiment, a pomegranate-comprising extract is added to ration for milking animal in an amount of 1 to 4 wt % (weight of extract per weight of dry ration), whereby improving the milk yield, and the milk quality when characterized by lowered number of somatic cells and increased antioxidative capacity. The increased production may be associated with lowered morbidity, improved digestion of the ration generally and of the fibers particularly, and in some cases with redirecting the metabolism from building the body mass to increasing the milk production, or it may involve other factors. In any case, the increase in the milk yield of milking animals, preferably cows, is in one embodiment of the method according to the invention between 1.5 and 15%. In some of the experiments, typical values have been between 2 and 12%.

In an important aspect of the invention, the method of improving the milk manufacturing process comprises adding CEP into ration whereby treating mastitis. Mastitis is usually treated by antibiotics, but such treatment may disqualify the milk for human consumption, whereas pomegranate components are acceptable in human diet without restrictions.

The invention will enable to increase ruminants' resistance to adverse conditions during their breeding, for example including weather conditions and bacterial infections, and will lower animals' morbidity and suffering. Simultaneously, the invention will substantially increase efficiency of the milk production and improve economy in dairy industry.

Provided by the invention is a pomegranate peel extract. It is believed by the inventors that the extract may be beneficial also for humans, for example for feeding lactating mothers. The invention relates to a food additive to be consumed by, and beneficial to mammals. In one embodiment, the consumption of said additive increases the milk production in mammal. In an industrially important aspect, the yield of milk in ruminants is substantially enhanced by said additive.

### EXAMPLES

### Example 1

The antioxidant activity in milk samples was determined by the chemiluminescence methods of Ginsburg I. et al. [Inflammopharmacology 12 (2004) 305-20]. Chemiluminescence was measured (counts per minute) in a standard mixture comprising glucose oxidase (GO), in a mixture further comprising a known amount of a compound with antioxidant activity (5 mM epicatechin), and in a mixture comprising either milk form the cows fed with the pomegranate enriched ration (PG) or milk from control cows. It can be seem that the “enriched” milk exhibits a signal closer to the epicatechin curve than the control milk. (see FIG. 1). The antioxidative capacity of the milk from cows fed with the pomegranate enriched ration (PG) was by 21% greater than the milk from control cows.

### Example 2

In one of experiments, six dairy cows were getting 500 ml aqueous extract from pomegranate peels (comprising 50 wt % of dry mass), supplied by mixing it directly with the daily ration. The daily ration of feed was 40 kg per cow containing 25 kg of dry matter. Simultaneously, six other cows were getting normal food without the extract, as control. During one week, the group of cows getting the “enriched” ration increased the milk yield; the experiment continued for two more weeks. The average milk yield of the group with enriched ration was higher by 7.3% than the yield of the control group over the last two weeks of the experiment. Beside the milk yield, the following parameters were determined for each cow at the beginning of every week: cow body weight, steps counting by pedometer, milk fat content, protein content, lactose content, and the total antioxidant activity in the milk. Not a substantial difference in the milk quality between the two groups was observed, except for the antioxidant activity, which were higher in the group fed on enriched fodder.

### Example 3

In another experiment, the influence of pomegranate peel extract on the number of somatic cells in raw milk was examined. Two Holstein dairy cows, 65 and 89 days after delivery, with very high count of somatic cells in milk received a pomegranate peel extract, while two other cows also with very high count of somatic cells did not get the extract, as a control. The peel extract was supplied to the cows as an additive to the normal feed in a an amount of 1.75 kg of liquid peel extract comprising 50 wt % of dry mass, directly admixed with the daily ration. The daily ration of feed was 40 kg per cow containing 25 kg of dry matter. The additive was added daily during 10 weeks. The somatic cell count was determined at the start and at the end of the test by using a somatic cell counter.

| | | Somatic cells per ml | Somatic cells per ml |
|---|---|---|---|
| | Additive | of raw milk at the start | of raw milk at the end |
| Cow 1 | Yes | 875,000 | 650,000 |
| Cow 2 | Yes | 730,000 | 495,000 |
| Cow 3 | No | 910,000 | 860,000 |
| Cow 4 | No | 689,000 | 715,000 |

As shown in the above table, the Somatic Cell Count decreased distinctly in the test group in comparison with the control group.

### Example 4

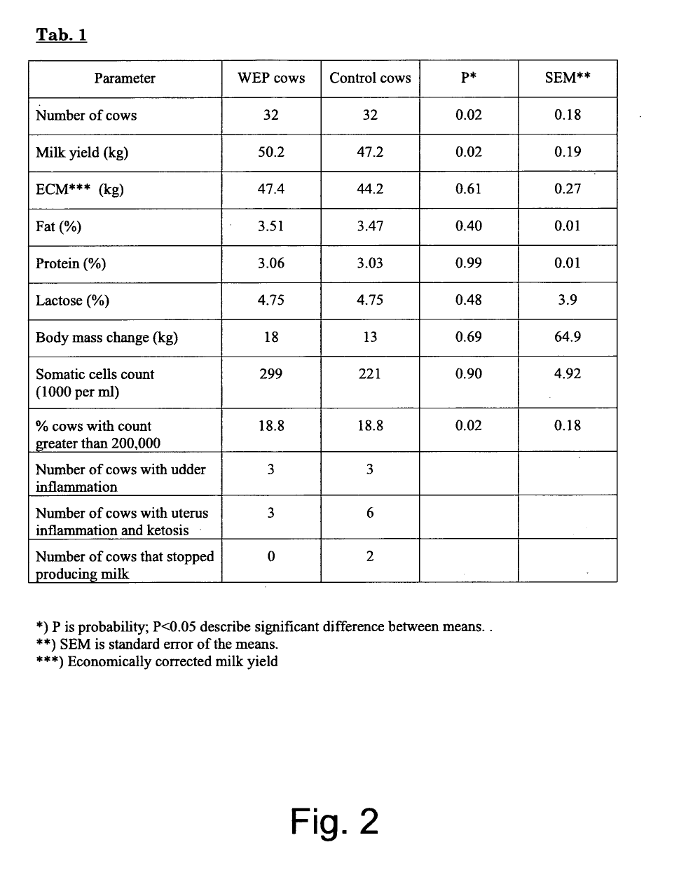

The goal of this experiment was to characterize effects of adding a CEP concentrate to the ration of milking cows on the milk yield, number of somatic cells in milk, antioxidative capacity of milk, milk composition, udder health, general health of the cow, and fodder consumption. The cows were checked regularly by a veterinary doctor. Samples from inflammation-afflicted udders were taken for microbiological tests. Two groups, 32 Holstein dairy cows each, were given CEP concentrate to ration in an amount of 4 wt % or 0 wt %, respectively (400 grams or nothing of CEP concentrate per 10 kg dry total mixed ration (TMR)). The addition of CEP to TMR started 20 days before calving and continued 80 days in milking after delivery, the control group did never get CEP. These cows (“calving cows”) usually have more health problems than cows more than 80 days after delivery (“normative cows”), and they have a negative energetic balance. The milk yield, composition, and body weight were monitored on daily basis. Somatic cell count was measured every two weeks during 90 days of the experiment. The health of udder was examined at the beginning and at the end of the experiment. Some of the results are in Table 1 (FIG. 2). The average milk yield was by 7.2% higher in the cows fed with pomegranate extract than in the control cows. The body mass change tended to be higher in the CEP-fed cows, and the number of cows with uterus inflammation or ketosis was reduced by 50%. Two cows in the control group stopped producing milk compared to zero in the CEP-fed cows.

### Example 5

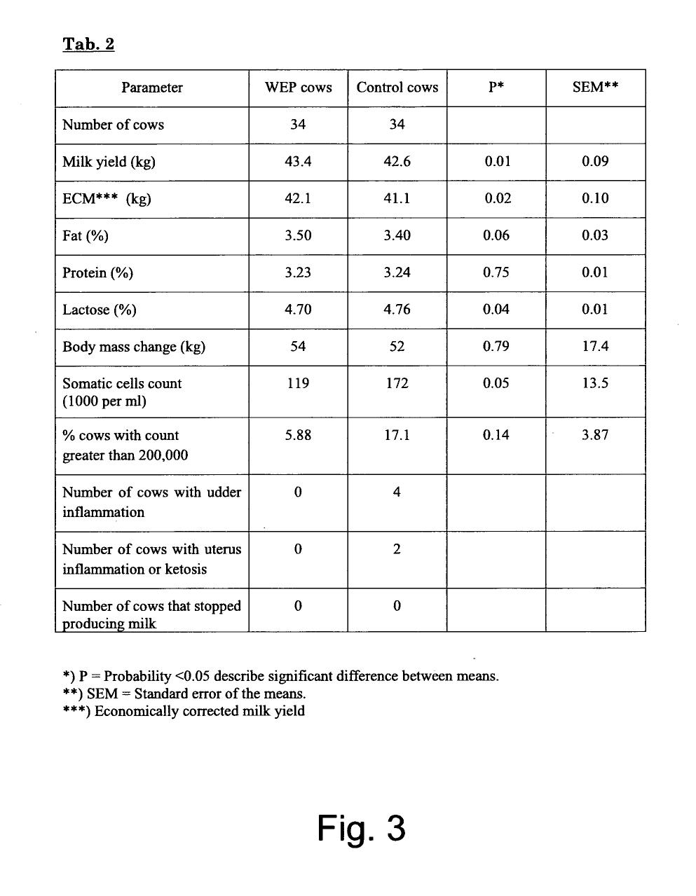

The goals of this experiment and the checked parameters were similar as in Example 4, but the cows included in the experiments were “normative” cows, it means cows more than 80 days after delivery that were healthy in terms of having SCC<150,000/ml milk. Two groups, 34 healthy Holstein dairy cows each, were given CEP concentrate to ration in an amount of 4 wt % or 0 wt %, respectively, the same as in Example 4, for 90 days. Some of the results are in Tab. 2 (FIG. 3). The average milk yield was just by 1.9% higher in the cows fed with pomegranate extract than in the control cows. But the main effect of CEP administration was reflected in cow's health and prevention of mastitis and inflammation. Proportion of cows with SCC in milk greater than 200,000/ml in the end of 90 days reduced from 17.1% in the control to 6.9% in the CEP group, while reducing average SCC by 31%. The proportion of cows with udder inflammation decreased from 11.8% in the control to 0 in the WEP group, and proportion of cows with uterus inflammation and ketosis reduced from 5.9% in the control to 0 in the CEP group.

### Example 6

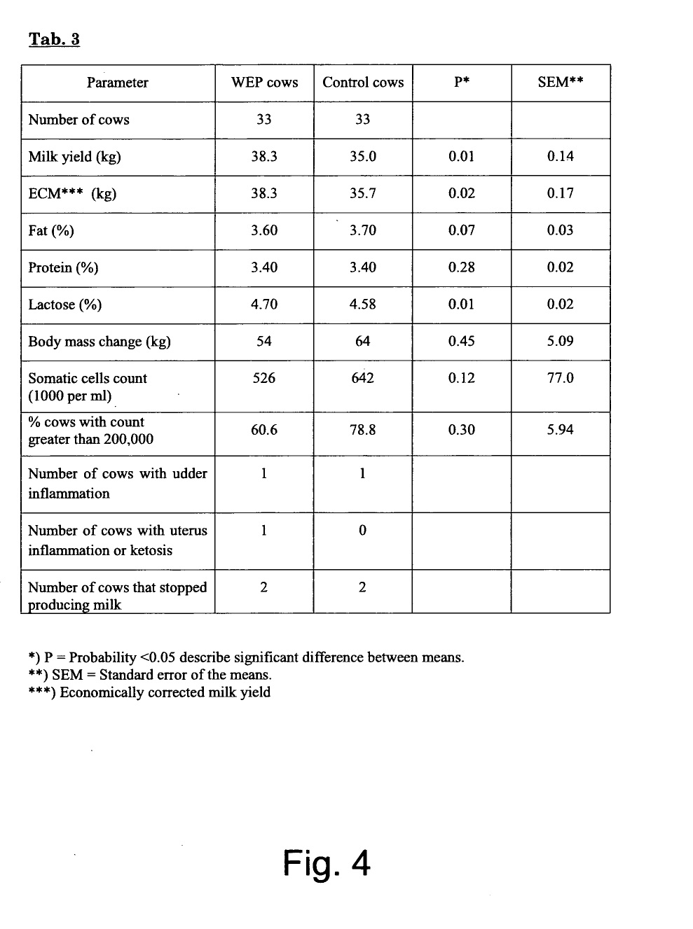

The goals of this experiment and the checked parameters were similar as in Example 5, but the cows included in the experiments were selected to have high somatic cells count from the beginning o the experiment (more than 150,000 cells per ml). Two groups of 33 Holstein dairy cows each having similar average milk yield at the beginning, were given CEP concentrate added to TMR to a level of 4 wt % or none for 90 days. Some of the results are in Tab. 3 (FIG. 4). The average milk yield was by 9.4% higher in the cows fed with pomegranate extract than in the control cows. The average SCC, and the proportion of cows with SCC>200,000 tended to be lower in the CEP group. It is concluded that the effect of CEP on the milk yield is higher in cows affected by health problems; this demonstrates the potential o the method of the invention for both increasing the milk production and for improving the animals' health.

### Example 7

The goal of this experiment was to characterize effects the CEP concentrate at various quantities added to the ration of milking cows. Examined parameters included the milk yield, ECM, SCC in milk, antioxidative capacity of milk, milk composition, voluntary dry matter intake of ration, efficiency (adjusted yield/intake), the dry matter digestibility of ration, fiber digestion (NDF), body mass changes, udder health, rumination time, and the recumbence time. Four groups, about 10 Holstein lactating dairy cows each, similar in the initial milk yield, were given CEP concentrate at a level of 0 wt %, 1 wt %, 2 wt %, 4 wt %, respectively (from zero to 400 grams of CEP concentrate per 10 kg dry ration) for six weeks. Voluntary dry matter intake, milk yield and composition, rumination time and recumbence time were monitored on a daily basis, and SCC on a weekly basis. Digestibility was measured based on non-digestible NDF in TMR and feces obtained from all cows through consequent four days of week 4 of the experiment. Antioxidative activity was measured in milk samples obtained from each individual cow at the onset of experiment and on week 5 of the experiment.

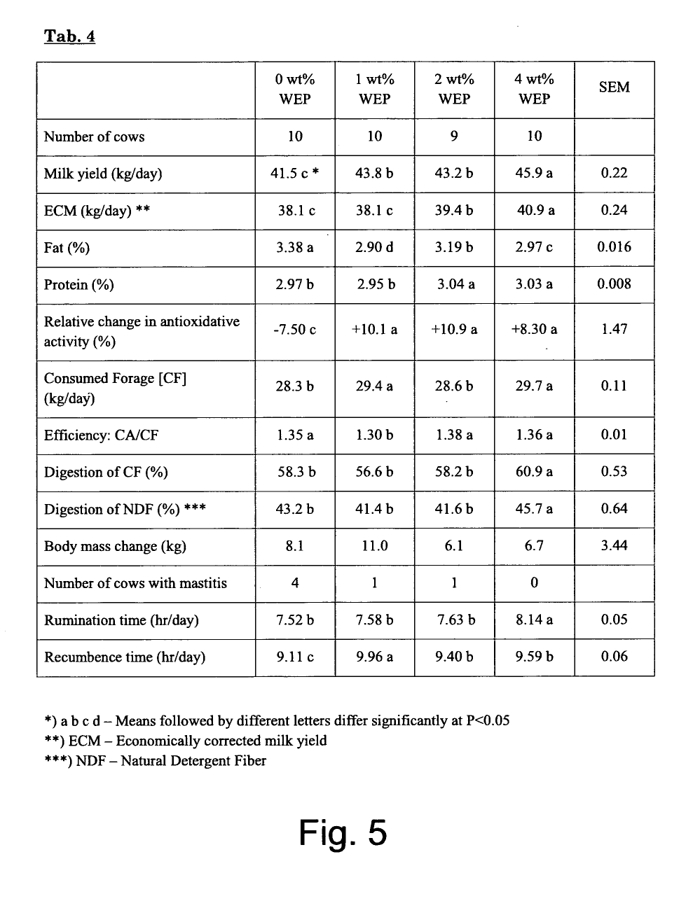

Since the level of antioxidative activity in milk decreased with time after calving, the data are given as the relative change between week 0 and week 5. Some of the results are in Tab. 4 (FIG. 5). The addition of a pomegranate-comprising extract has improved the milk yield in any amount of CEP addition between 1 and 4 wt %. The addition of 4 wt % resulted in the milk production increase by 10.6%. The increased production seems to be associated with lowered morbidity, improved digestion of the ration generally and of the fibers particularly, as well as with redirecting the metabolism from building the body mass to increasing the milk production. The udder inflammation (mastitis) nearly disappeared in all the cows which were administered the CEP in any concentration between 1 and 4 wt %, even though cattle was largely afflicted by E. coli-caused mastitis in the site and during the time of the experiments (40% animals of the control animals, not-protected by CEP, were afflicted). Mastitis was treated by antibiotics in all groups. The addition of 4 wt % CEP concentrate led to the increase in the average daily milk production of 10.6% in this experiment.

### Example 8

This example shows one embodiment of the method according to the invention of preparing a pomegranate aqueous extract to be added to animal's fodder to enhance the quantity and quality of milk. 100 tons of pomegranate fruit from the Wonderful variety growing in Israel were processed to obtain the juice by squeezing. The residual matter after squeezing the fruit, about 68 tons, was mixed with tap water at 60° C., three weight parts of water per one weight part of the pomegranate matter. After a soaking period of 4 hours, with slow stirring, the water extract was separated from the insoluble suspended fragments by filtration and concentrated in a vacuum evaporator at a temperature of up to 70° C. 12 tons of pomegranate aqueous concentrate 48.0 Brix, as determined by refractometer, were obtained. The extract was used in above Examples 4-7.

Analytical values of the extract were determined, as shown in the following table, wherein CAA stands for is citric acid anhydrous, ORAC stands for oxygen radical absorbance capacity, TE stands for trolox equivalent:

| Parameter | Unit | Analysis |
|---|---|---|
| Brix - by refractometer | ° | 48 |
| Acidity | w/v % as CAA | 3.88 |
| pH | | 3.55 |
| Punicalagins A + B, by HPLC | mg/Kg | 26,500 |
| Ellagic acid, by HPLC | mg/Kg | 2,500 |
| Total phenolics calculated as gallic acid, | mg/Kg | 92,000 |
| by Folin-Ciocalteu | | |
| Hydrophilic ORAC value | μM TE/100 g | 48,000 |

While the invention has been described using some specific examples, many modifications and variations are possible. It is therefore understood that the invention is not intended to be limited in any way, other than by the scope of the appended claims.

1. A method for enhancing the quantity and quality of milk in a mammal, while maintaining or enhancing its health, comprising feeding said mammal with a ration comprising an additive derived from pomegranate plant.

2. A method according to claim 1, wherein said mammal is a ruminant.

3. A method according to claim 1, wherein said additive is prepared by processing a pomegranate part selected from the group consisting of leaves, flowers, fruits, fruit cores, fruit peels, fruit pulp membranes, fruit arils, and mixtures thereof.

4. A method according to claim 3, wherein said additive comprises an active compound in a concentration higher than the concentration of said compound in said pomegranate part before said processing.

5. A method according to claim 3 for enhancing the yield of milk obtained from ruminants, comprising adding to the ration for said ruminants an additive comprising pomegranate peels selected from the group consisting of dried pomegranate parts, an extract from pomegranate parts, and silage of pomegranate parts.

6. A method according to claim 5 for enhancing the yield of milk, wherein said ruminants are dairy cattle, comprising adding to the ration for said cattle dry pomegranate peels or an extract of pomegranate peels.

7. A method according to claim 5, wherein said extract is an aqueous extract or an alcohol-based extract, and it comprises from 1 to 100 wt % of dry mass.

8. A method according to claim 5, wherein said step of adding the additive to ration results in lowering the number of somatic cells in the obtained milk.

9. A method according to claim 5, wherein said step of adding the additive to ration results in a higher content of antioxidants in the obtained milk.

10. A method according to claim 5, wherein the milk yield is increased by 1.5% or more after including said additive in said ration.

11. A method according to claim 10, wherein said milk yield is increased by 3.0% or more.

12. A method according to claim 11, wherein said milk yield is increased by 6.0% or more.

13. A method according to claim 1, wherein said feeding with a ration comprising a pomegranate-derived additive results in improving animals' well being and health.

14. A method according to claim 13, comprising lowering morbidity associated with a disease selected from the group consisting of a bacterial infection, mastitis, uterus inflammation, and ketosis.

15. A method according to claim 1, wherein said feeding with a ration comprising a pomegranate-derived additive results in improving at least one of the parameters selected from the group consisting of the number of somatic cells in milk, antioxidative activity of milk, milk composition, voluntary dry matter intake, dry matter digestibility, NDF digestibility, body mass changes, udder health, general health of the animal, the rumination time, and the recumbence time.

16. A method for improving efficiency of the milk production in dairy industry, comprising feeding milking ruminants with a fodder consisting of a ration and a pomegranate-derived additive, thereby
i) increasing the daily milk yield of said ruminants,
ii) improving the milk quality, and
iii) enhancing health and well being of said ruminants.

17. A method according to claim 16, comprising enhancing health and well being of said ruminants under stress conditions selected from ambient heat load, beginning of lactation, bacterial infection, inflammation of uterus, and inflammation of udder.

18. A food additive for increasing the milk yield in ruminants, comprising processed pomegranate parts selected from leaves, flowers, fruits, fruit cores, fruit peels, fruit pulp membranes, fruit arils, and mixtures thereof, wherein processing said parts comprises a step selected from drying, extracting, and ensiling.

19. A food additive according to claim 18, comprising an aqueous extract from pomegranate peels.

20. A food additive according to claim 18, comprising an alcoholic extract from pomegranate peels.

21. A food additive according to claim 18, comprising a concentrated aqueous extract from pomegranate peels.

22. A food additive according to claim 18, comprising a silage from pomegranate-derived parts containing peels.

23. A food additive according to claim 18, wherein said extract comprises at least one characteristic selected from the group consisting of punicalagins A+B in an amount of more than 2,000 mg/Kg, total phenolics determined by Folin-Ciocalteu reagent in an amount of more than 10,000 mg/Kg, and ORAC value of more than 5,000 μm TE/100 g.

24. An enriched fodder for a ruminant, comprising a suitable ration and the food additive of claim 18, the additive constituting from 0.5 to 20 weight parts per 100 dry weight parts of said ration.

25. A process for manufacturing the additive of claim 18, comprising mixing the pomegranate peels or parts containing them with a solvent acceptable in animal husbandry, thereby obtaining a suspension of insoluble fragments in liquid, separating said suspended fragments from said liquid, and removing at least a part of said solvent from the mixture.

26. A process according to claim 25, wherein said solvent is selected from the group consisting of water, alcohol, or a mixture thereof, and said separating comprises sieving, filtering, or centrifuging.

27. A process according to claim 25, further comprising determining at least one characteristic selected from the group consisting of punicalagins, total phenolics, ORAC value, and ellagic acid.
